(19) United States
(12) Patent Application Publication
(10) Pub. No.: US 2026/0007362 A1
(43) Pub. Date: Jan. 8, 2026

(54) TECHNIQUES FOR DETECTING, RECORDING, AND REGENERATING DREAMS

(71) Applicant: Oura Health Oy, Oulu (FI)

(72) Inventors: Mika Erkkilä, Oulu (FI); Mats Kyrrö, Oulu (FI)

(21) Appl. No.: 18/766,428

(22) Filed: Jul. 8, 2024

(51) Int. Cl. A61B 5/00

(52) U.S. Cl. CPC A61B 5/4812; A61B 5/6802; A61B 5/7264; A61B 5/7435; A61B 5/746

(57) ABSTRACT

Methods, systems, and devices for recording dream characterizations are described. A system may receive physiological data measured from a user and may compute one or more sleep stages of the user, including a first sleep cycle, based on the physiological data. The system may detect, based on the physiological data, that the user has woken up within a first threshold duration from an end of the first sleep cycle and may display information prompting the user to input one or more characteristics associated with a dream based on detecting the user has woken up within the first threshold duration from the end of the first sleep cycle. Additionally, the system may generate, using a generative artificial intelligence model, one or more of a visual representation, a textual representation, or an auditory representation of the dream based on the one or more characteristics input by the user.

### FIELD OF TECHNOLOGY

The following relates to wearable devices and data processing, including techniques for detecting, recording, and regenerating dreams.

### BACKGROUND

Some wearable devices may be configured to collect data from users associated with movement and other activities. For example, some wearable devices may be configured to detect when a user is asleep. However, many users may dream during their sleep and conventional systems may be unable to detect when a user is dreaming and record data associated with a dream.

### BRIEF DESCRIPTION OF THE DRAWINGS

FIG. 1 illustrates an example of a system that supports techniques for detecting, recording, and regenerating dreams in accordance with aspects of the present disclosure.

FIG. 2 illustrates an example of a system that supports techniques for detecting, recording, and regenerating dreams in accordance with aspects of the present disclosure.

FIG. 3 shows an example of a system that supports techniques for detecting, recording, and regenerating dreams in accordance with aspects of the present disclosure.

FIG. 4 shows an example of a graphical user interface (GUI) that supports techniques for detecting, recording, and regenerating dreams in accordance with aspects of the present disclosure.

### DETAILED DESCRIPTION

In some systems, a wearable device may be configured to collect physiological data associated with a user, such as sleep data, and may display, via a user device, information back to the user regarding their sleep. For example, a system may detect that that a user slept from 9:30 pm to 6:30 am and may display information back to the user regarding their sleep between 9:30 pm and 6:30 am, such as a duration of their sleep, movement during their sleep, an average heart rate during their sleep, or the like thereof. However, many users may dream during their sleep and conventional systems may be unable to detect when a user is dreaming (e.g., or a likelihood that they are dreaming) and may be unable to record data associated with a dream. Additionally, or alternatively, a user may dream well before (e.g., a first threshold duration before) they wake up such that, when the user wakes up, the user may not recall that they dreamt or may not be able to recall contents of their dream.

Accordingly, techniques described herein may enable a system to identify an interval during which a user may have dreamt (e.g., identify the user likely dreamt), prompt the user to record a dream after waking up (e.g., at an end of or within a second threshold duration after the interval), and display a representation of the dream to the user based on the information recorded by the user about the dream. For example, the system may include a wearable device that collects physiological data associated with a user, including at least sleep data, such that the system may identify multiple sleep stages of the user based on the physiological data (e.g., sleep data), including at least a first sleep cycle (e.g., rapid eye movement (REM) cycle) of the user. Additionally, the system may identify that the user has woken up at or close to (e.g., within the second threshold duration of) the end of the first sleep cycle and may prompt the user to record information associated with a dream based on detecting the user woke up at or close to the end of the first cycle.

In some examples, the user may wake up naturally, such that the system may detect the user has woken up based on the physiological data indicating the user is awake. In some other examples, the system may generate an alarm, via the wearable device, the user device, an external device, or any combination thereof, to wake the user up, where the alarm is an auditory alarm, a haptic alarm, and a visual alarm, or any combination thereof. In some cases, the system may generate the alarm based on detecting that the user is within the second threshold duration of the end of the first sleep cycle (e.g., no more than 5 minutes after the end of the first sleep cycle), based on a duration of the first sleep cycle being within a third threshold of an average first sleep cycle of the user (e.g., the first sleep cycle is 35 minutes and an average first sleep cycle is 37 minutes), based on the duration of the first sleep cycle exceeding a fourth threshold duration (e.g., a user may need to sleep at least 20 minutes in the first sleep cycle to dream), or any combination thereof. Additionally, or alternatively, the system may generate the alarm based on a change in physiological data of the user (e.g., an increase in heart rate, an increase in temperature, or the like thereof, may indicate the user is dreaming). Additionally, or alternatively, the system may include an external device capable of measuring brain wave data of the user, such that the system may generate the alarm based on on a frequency of the brain wave data exceeding a threshold frequency (e.g., an increase in high-frequency brain waves may indicate the user dreaming).

To record the information associated with the dream, a graphical user interface (GUI) of a user device associated with the wearable device may display information prompting the user to input one or more characteristics (e.g., characterizations) associated with the dream. In some cases, the GUI may automatically prompt the user to input the one or more characteristics based on detecting that the user has woken up. In some other cases, the GUI may prompt the user to input the one or more characteristics based on receiving a user input (e.g., indicating the user has a dream to record). In some examples, the user may input the one or more characteristics by selecting one or more tags (e.g., visual or textual) associated with the dream, may verbally input a description of the dream (e.g., with or without artificial intelligence (AI) assistance), may textually input the description of the dream (e.g., with or without AI assistance), or any combination thereof. For example, the user may recite a set of words or broken sentences describing the dream, such that the system, using AI assistance, may generate one or more complete sentences describing the dream based on the set of works or broken sentences. In such cases, the system may enable the user to edit the one or more complete sentences generated using AI assistance.

Additionally, the system may generate, using an AI model, one or more of a visual representation, a textual representation, or an auditory representation of the dream based on the one or more characteristics input by the user. For example, the one or more characteristics input by the user may include a description of the user driving a car on a sunny day, such that the system may generate, using the AI model, a short video of the user driving the car on the sunny day. In such cases, the system may user one or more characteristics of the user (e.g., age, sex, height, hair color, or the like thereof) to generate a visual depiction of the user in the short video. Additionally, the system may prompt the user to input feedback associated with the visual representation, the textual representation, the auditory representation, or any combination thereof. For example, the video generated by the system may depict a red car, however, the user may have dreamt of a blue car, such that the user may indicate the difference in car color to the system. Thus, the system may receive the feedback and may update the visual representation, the textual representation, the auditory representation, or any combination thereof based on the feedback. For example, the system may update the video to depict the blue car rather than the red car.

In some cases, the system may identify one or more trends associated with the user dreaming. That is, the system may identify one or more trends between multiple dream occurrences (e.g., and/or respective dream characteristics) and at least one of physiological data associated with the user, lifestyle data associated with the user (e.g., tags input by the user, activities performed by the user, current events, food consumed by the user, or the like thereof), and respective dream characteristics. For example, the system may identify that the user is likely to dream after consuming a high fat dinner based on identifying a threshold quantity of dream occurrences occurring after the user has consumed a high fat dinner. In another example, the system may identify that when a stress level of the user exceeds a threshold stress level, the user is likely to dream about current events or a source of the stress.

Aspects of the disclosure are initially described in the context of systems supporting physiological data collection from users via wearable devices. Aspects of the disclosure are then described in the context of a GUI. Aspects of the disclosure are further illustrated by and described with reference to apparatus diagrams, system diagrams, and flowcharts that relate to techniques for detecting, recording, and regenerating dreams.

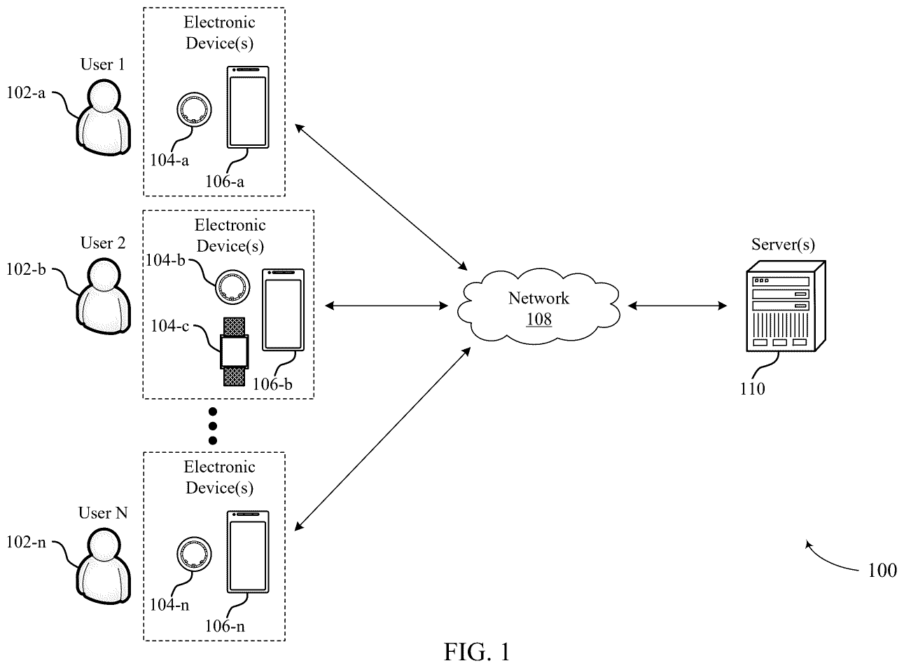

FIG. 1 illustrates an example of a system 100 that supports techniques for detecting, recording, and regenerating dreams in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for identifying an interval during which a user 102 may have dreamt, prompting the user 102 to record a dream upon waking up (e.g., at an end of or within a first threshold duration after the interval), and display a representation of the dream to the user 102 based on the information recorded by the user 102 about the dream. For example, a ring 104 of the system 100 may collect physiological data associated with a user 102, including at least sleep data, such that the system 100 may identify multiple sleep stages of the user 102 based on the physiological data (e.g., sleep data), including at least a first sleep cycle (e.g., REM cycle) of the user 102. Additionally, the system 100 may identify that the user 102 has woken up at or close to (e.g., within the first threshold duration of) the end of the first sleep cycle and may prompt the user 102 to record information associated with a dream via a user device 106 based on detecting the user 102 woke up at or close to the end of the first cycle.

In some examples, the user 102 may wake up naturally, such that the system 100 may detect the user 102 has woken up based on the physiological data indicating the user 102 is awake. In some other examples, the system 100 may generate an alarm, via the ring 104, the user device 106, an external device (e.g., a watch 104, an alarm clock connected to the network 108), or any combination thereof, to wake the user 102 up, where the alarm is an auditory alarm (e.g., beeping, alarm sound), a haptic alarm (e.g., vibration), and a visual alarm (e.g., turning lights on, flashing), or any combination thereof.

To record the information associated with the dream, a GUI of the user device 106 associated with the ring 104 may display information prompting the user 102 to input one or more characteristics (e.g., characterizations) associated with the dream. In some cases, the GUI may automatically prompt the user 102 to input the one or more characteristics based on detecting that the user 102 has woken up. In some other cases, the GUI may prompt the user 102 to input the one or more characteristics based on receiving a user input (e.g., indicating the user 102 has a dream to record) via the user device 106.

In some examples, the user 102 may input the one or more characteristics by selecting one or more tags (e.g., visual or textual) associated with the dream, may verbally input a description of the dream (e.g., with or without AI assistance), may textually input the description of the dream (e.g., with or without AI assistance), or any combination thereof. For example, the user 102 may recite a set of words or broken sentences describing the dream, such that the system 100, using AI assistance, may generate one or more complete sentences describing the dream based on the set of works or broken sentences. In such cases, the system 100 may enable the user 102 to edit the one or more complete sentences generated using AI assistance.

Additionally, the system 100 may generate, using an AI model, one or more of a visual representation, a textual representation, or an auditory representation of the dream based on the one or more characteristics input by the user 102. For example, the one or more characteristics input by the user 102 may include a description of the user 102 driving a car on a sunny day, such that the system 100 may generate, using the AI model, a short video of the user 102 driving the car on the sunny day. Additionally, the system 100 may prompt the user 102 to input feedback associated with the visual representation, the textual representation, the auditory representation, or any combination thereof. For example, the video generated by the system 100 may depict a red car, however, the user 102 may have dreamt of a blue car, such that the user 102 may indicate, via the user device 106, the difference in car color to the system 100. Thus, the system 100 may receive the feedback and may update the visual representation, the textual representation, the auditory representation, or any combination thereof based on the feedback. For example, the system 100 may update the video to depict the blue car rather than the red car.

It should be appreciated by a person skilled in the art that one or more aspects of the disclosure may be implemented in a system 100 to additionally or alternatively solve other problems than those described herein. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

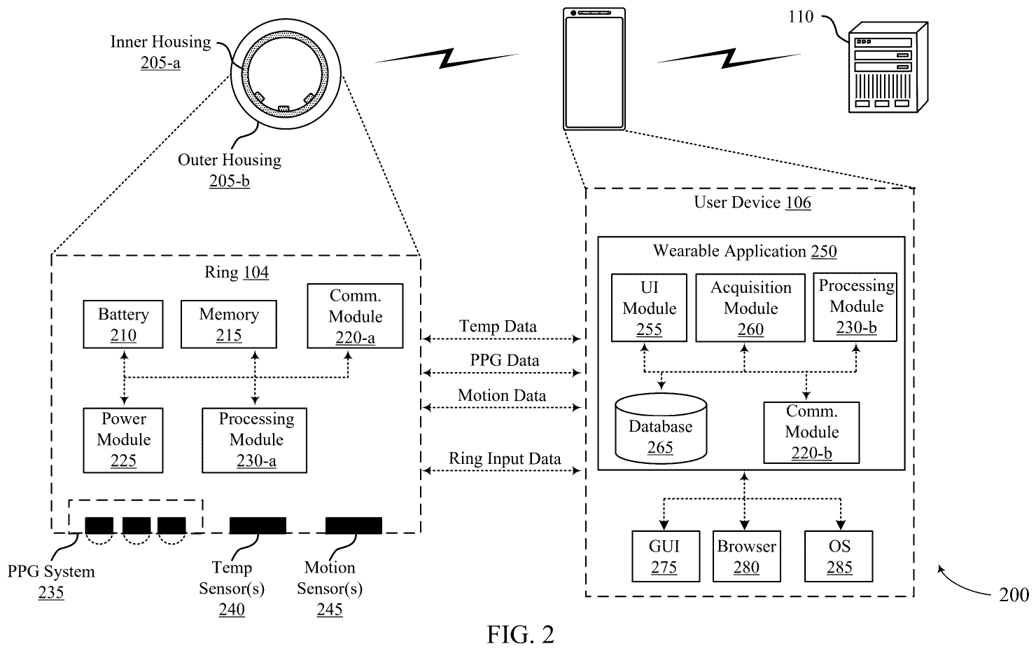

FIG. 2 illustrates an example of a system 200 that supports techniques for detecting, recording, and regenerating dreams in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques for identifying an interval during which a user 102 may have dreamt, prompting the user 102 to record a dream upon (e.g., after) waking up (e.g., at an end of or within a first threshold duration after the interval), and display a representation of the dream to the user 102 based on the information recorded by the user 102 about the dream. For example, a ring 104 of the system 200 may collect physiological data associated with a user 102, including at least sleep data, such that the system 200 may identify multiple sleep stages of the user 102 based on the physiological data (e.g., sleep data), including at least a first sleep cycle (e.g., REM cycle) of the user 102. Additionally, the system 200 may identify that the user 102 has woken up at or close to (e.g., within the first threshold duration of) the end of the first sleep cycle and may prompt the user 102 to record information associated with a dream via a user device 106 based on detecting the user 102 woke up at or close to the end of the first cycle.

In some examples, the user 102 may wake up naturally, such that the system 200 may detect the user 102 has woken up based on the physiological data indicating the user 102 is awake. In some other examples, the system 200 may generate an alarm, via the ring 104, the user device 106, an external device (e.g., a watch 104, an alarm clock connected to the network 108), or any combination thereof, to wake the user 102 up, where the alarm is an auditory alarm (e.g., beeping, alarm sound), a haptic alarm (e.g., vibration), and a visual alarm (e.g., turning lights on, flashing), or any combination thereof. In some cases, the system 200 may generate the alarm based on detecting that the user 102 is within the first threshold duration of the end of the first sleep cycle (e.g., no more than 5 minutes after the end of the first sleep cycle), based on a duration of the first sleep cycle being within a second threshold of an average first sleep cycle of the user 102 (e.g., the first sleep cycle is 35 minutes and an average first sleep cycle is 37 minutes), based on the duration of the first sleep cycle exceeding a third threshold duration (e.g., a user may need to sleep at least 20 minutes in the first sleep cycle to dream), or any combination thereof. Additionally, or alternatively, the system 200 may generate the alarm based on a change in physiological data of the user 102. For example, the system 200 may detect an increase in heart rate based on physiological data collected via the PPG system 235, may detect an increase in temperature of the user 102 based on physiological data collected via the temperature sensors 240, or the like like thereof, which may be indicative that the user 102 is dreaming Additionally, or alternatively, the system 200 may include an external device capable of measuring brain wave data of the user, such that the system 200 may generate the alarm based on a frequency of the brain wave data exceeding a threshold frequency (e.g., an increase in brain wave frequency may indicate the user dreaming).

To record the information associated with the dream, a GUI 275 of the user device 106 associated with the ring 104 may display information prompting the user 102 to input one or more characteristics (e.g., characterizations) associated with the dream. In some cases, the GUI 275 may automatically prompt the user 102 to input the one or more characteristics based on detecting that the user 102 has woken up. In some other cases, the GUI 275 may prompt the user 102 to input the one or more characteristics based on receiving a user input (e.g., indicating the user 102 has a dream to record) via the user device 106.

In some examples, the user 102 may input the one or more characteristics by selecting one or more tags (e.g., visual or textual) associated with the dream, may verbally input a description of the dream (e.g., with or without AI assistance), may textually input the description of the dream (e.g., with or without AI assistance), or any combination thereof. For example, the user 102 may recite a set of words or broken sentences describing the dream, such that the system 200, using AI assistance, may generate one or more complete sentences describing the dream based on the set of works or broken sentences. In such cases, the system 200 may enable the user 102 to edit the one or more complete sentences generated using AI assistance.

Additionally, the system 200 may generate, using an AI model, one or more of a visual representation, a textual representation, or an auditory representation of the dream based on the one or more characteristics input by the user 102. For example, the one or more characteristics input by the user 102 may include a description of the user 102 driving a car on a sunny day, such that the system 200 may generate, using the AI model, a short video of the user 102 driving the car on the sunny day and may display the video to the user via the GUI 275 Additionally, the system 200 may prompt the user 102 to input feedback associated with the visual representation, the textual representation, the auditory representation, or any combination thereof. For example, the video generated by the system 200 may depict a red car, however, the user 102 may have dreamt of a blue car, such that the user 102 may indicate, via the user device 106, the difference in car color to the system 200. Thus, the system 200 may receive the feedback and may update the visual representation, the textual representation, the auditory representation, or any combination thereof based on the feedback. For example, the system 200 may update the video to depict the blue car rather than the red car.

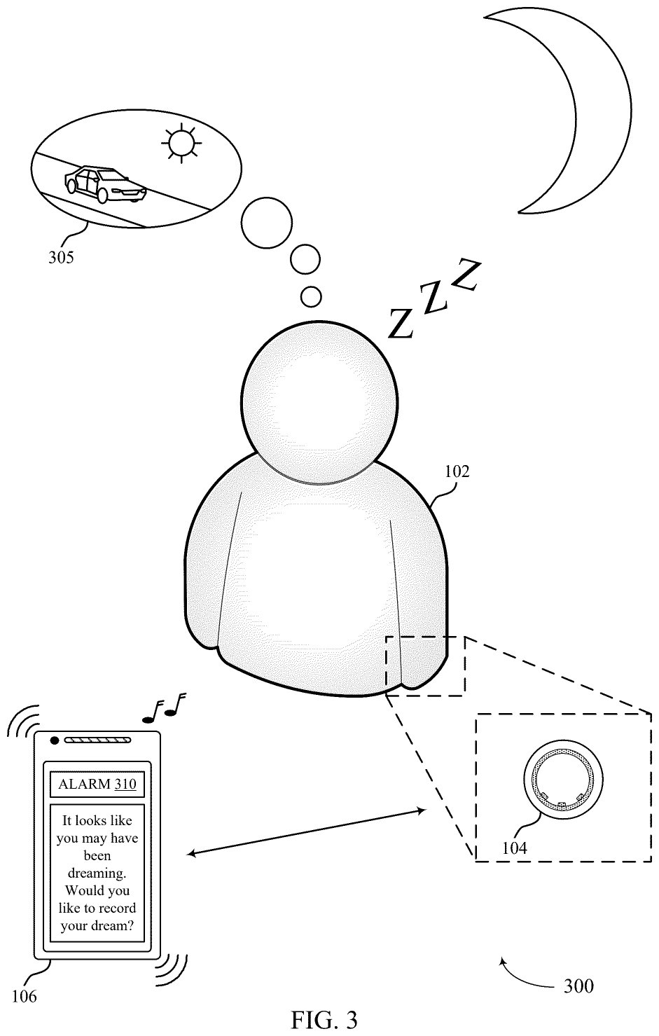

FIG. 3 shows an example of a system 300 that supports techniques for detecting, recording, and regenerating dreams in accordance with aspects of the present disclosure.

Some wearable devices may be configured to collect physiological data associated with a user 102, such as sleep data, and may display, via a user device 106, information back to the user regarding their sleep. For example, a system may detect that that a user 102 slept from 9:30 pm to 6:30 am and may display information back to the user 102 regarding their sleep between 9:30 pm and 6:30 am, such as a duration of their sleep, movement during their sleep, an average heart rate during their sleep, or the like thereof. However, many users 102 may dream during their sleep and conventional systems may be unable to detect when a user 102 is dreaming (e.g., or a likelihood that they are dreaming) and record data associated with a dream. Additionally, or alternatively, a user 102 may dream well before (e.g., a first threshold duration before) they wake up such that, when the user 102 wakes up, the user 102 may not recall that they dreamt or may not be able to recall contents of their dream. For example, the user 102 who slept from 9:30 pm to 6:30 am may experience a dream 305 in the middle of the night (e.g., 12:00 pm), however, the user 102 may not wake up until 6:30 am and may not recall that they dreamt, or may not what they dreamt about, based on 6.5 hours occurring between when the user 102 dreamt and when the user 102 woke up.

Accordingly, techniques described herein may enable the system 300 to identify that a user 102 is dreaming (e.g., is likely to be dreaming or is likely to have dreamt), prompt the user 102 to record a dream 305 after waking up (e.g., at an end of or within a second threshold duration after an interval during which the user 102 was dreaming or was likely to have been dreaming), and display a representation of the dream 305 to the user 102 based on the information recorded by the user 102 about the dream. For example, the system 300 may include a wearable device, such as a ring 104, that collects physiological data associated with a user 102, including at least sleep data, such that the system 300 may identify multiple sleep stages of the user 102 based on the physiological data (e.g., sleep data), as described with reference to FIG. 1. In such cases, the multiple sleep stages of the user 102 may include, at least, a first sleep cycle of the user, where the first sleep cycle may be, for example, a REM cycle, a non-REM cycle, or the like thereof.

Additionally, the system 300 may identify that the user 102 is dreaming or is likely dreaming (e.g., or is likely to have dreamt). In some cases, the system 300 may identify that the user is likely dreaming or is likely to have dreamt based on the first sleep cycle of the user 102. For example, REM cycles may be associated with a higher likelihood of dreaming than other sleep stages of the multiple sleep stages, such that the system 300 may identify that the user 102 is likely dreaming (e.g., or is likely to have dreamt) based on identifying a REM cycle of the user 102. Additionally, or alternatively, the system 300 may identify that the user 102 is dreaming (e.g., is likely dreaming) based on physiological data associated with the user 102. For example, any combination of an increase in heart rate (e.g., coupled with a low HRV), an increase in temperature, or the like thereof may be indicative of the user 102 dreaming. Additionally, or alternatively, the system 300 may include an external device, such as an electroencephalogram (EEG), configured to acquire brain wave data associated with the user 102, such that the system 300 may identify that the user 102 is dreaming based on the brain wave data. For example, an increase in high-frequency brain wave data (e.g., an increase in high-frequency brain waves) may be indicative of the user 102 dreaming.

Additionally, the system 300 may identify that the user 102 has woken up after dreaming (e.g., after the user 102 has likely dream) or within a second threshold duration of dreaming and may prompt the user 102 to record information associated with a dream. For example, in some cases, the system 300 may identify that the user 102 has woken up at or close to (e.g., within the second threshold duration of) the first sleep cycle (e.g., REM cycle) and may prompt the user 102 to record information associated with a dream 305 based on detecting the user 102 woke up at or close to the end of the first sleep cycle (e.g., based on a likelihood that the user 102 dreamt during the first sleep cycle). Additionally, or alternatively, the system 300 may identify that the user 102 has woken up while experiencing or shortly after experiencing (e.g., within the second threshold duration of experiencing) a change in physiological data (e.g., an increase in heart rate, an increase in temperature) and may prompt the user 102 to record information associated with a dream 305 based on detecting the user 102 woke up while experiencing or shortly after experiencing the change in physiological data (e.g., based on the change in physiological data indicating that the user 102 is likely dreaming). Additionally, or alternatively, the system 300 may identify that the user 102 has woken up while experiencing or shortly after experiencing (e.g., within the second threshold duration of experiencing) a change in brain wave data (e.g., an increase in high-frequency brain waves) and may prompt the user 102 to record information associated with a dream 305 based on detecting the user 102 woke up while experiencing or shortly after experiencing the change in brain wave data (e.g., based on the change in brain wave data indicating that the user 102 is likely dreaming). Other data associated with the user 102 that may be used by the system 300 to detect that the user 102 is dreaming (e.g., or not dreaming) may include body movement (e.g., an increase in body movement may indicate dreaming), eye movement (e.g., an increase in eye movement may indicate dreaming), snoring (e.g., snoring may indicate the user 102 is not dreaming), or the like thereof.

In some examples, the user 102 may wake up naturally, such that the system 300 may detect the user 102 has woken up based on the physiological data indicating the user is awake. In some other examples, the system 300 may generate an alarm 310, via the ring 104, a user device 106 associated with the ring 104, an external device, or any combination thereof, to wake the user 102 up, where the alarm 310 is an auditory alarm 310, a haptic alarm 310, and a visual alarm 310, or any combination thereof. For example, the system 300 may generate a first signal to cause the user device 106 to emit a pre-selected (e.g., or default) alarm sound, may generate a second signal to cause the ring 104 to vibrate, may generate a third signal to cause lights in a room of the user 102 to turn on, or any combination thereof.

In such cases, the system 300 may generate the alarm 310 based on detecting (e.g., identifying) that the user 102 is dreaming or is likely dreaming (e.g., or is likely to have dreamt). For example, in some cases, the system 300 may generate the alarm 310 based on detecting that the user 102 is within the second threshold duration of the end of the first sleep cycle (e.g., at an end of the first sleep cycle or no more than 5 minutes after the end of the first sleep cycle), based on a duration of the first sleep cycle being within a third threshold of an average first sleep cycle of the user (e.g., the first sleep cycle is 35 minutes and an average first sleep cycle is 37 minutes), based on the duration of the first sleep cycle exceeding a fourth threshold duration (e.g., a user 102 may need to sleep at least 20 minutes in the first sleep cycle to dream), or any combination thereof. Additionally, or alternatively, the system 300 may generate the alarm 310 based on one or more changes in physiological data of the user 102 (e.g., the increase in heart rate, the increase in temperature, or the like thereof, may indicate the user is dreaming). For example, the system 300 may identify an increase in heart rate of the user 102 (e.g., a first change in physiological data) and may generate the alarm 310 based on identifying that the heart rate of the user 102 is beginning to decrease after the increase (e.g., a second change in physiological data). That is, the system 300 may generate the alarm 310 within the second threshold duration of the decrease in heart rate (e.g., which may indicate the user 102 has finished dreaming). Additionally, or alternatively, the system 300 may generate the alarm 310 based on one or more changes in frequency of the brain wave data. For example, the system 300 may identify that the frequency of the brain wave data of the user 102 has exceeded a threshold frequency (e.g., indicative of the user 102 dreaming) and may generate the alarm 310 within the second threshold duration of the frequency of the brain wave data of the user 102 falling back below the threshold frequency (e.g., indicative that the user 102 is done dreaming).

In some cases, the user 102 may enable or disable the capability of the system 300 to generate alarms (e.g., an alarm feature). For example, the user 102 may experience a tiring day and may desire to not be woken up while they sleep, even if they have dreamt, such that the user 102 may disable the alarm feature. In some cases, activating a rest mode associated with the system 300 may automatically disable the alarm feature. Additionally, or alternatively, the system 300 may generate an alarm based on satisfaction of one or more criteria. For example, the one or more criteria may be based on the sleep data of the user 102 (e.g., if the user 102 is experiencing poor sleep or experienced poor sleep a previous night, the system 300 may disable alarms), activity data associated with the user 102 during a previous day (e.g., if the user 102 was very active such that rest is beneficial for the user 102, the system 300 may disable alarms), the user being within a fifth threshold duration of an average wake up time of the user 102 (e.g., the system 300 may only wake the user 102 if they are within 30 minutes of an average wake up time of the user 102), or any combination thereof. Additionally, or alternative, the user 102 may select one or more settings associated with alarms, such as a type of alarm (e.g., visual, haptic, or auditory), the second threshold duration (e.g., whether to wake the user 102 immediately after dreaming or during light sleep), or the like thereof.

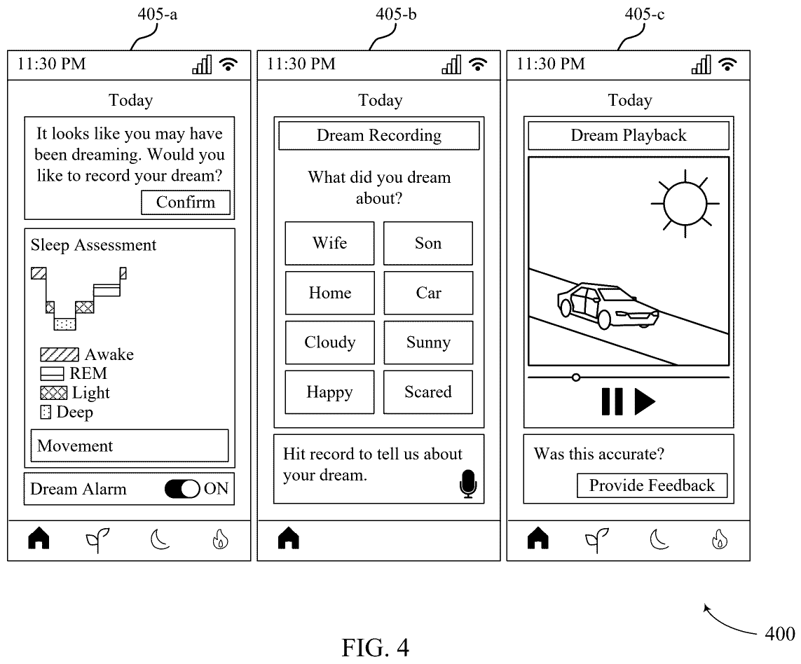

To record the information associated with the dream 305, the system 300 may generate a signal to cause a GUI of the user device 106 associated with the ring 104 to display information prompting the user 102 to input one or more characteristics (e.g., characterizations) associated with the dream 305, as described further with reference to FIG. 4. Additionally, the system 300 may generate, using an AI model, one or more of a visual representation, a textual representation, or an auditory representation of the dream 305 based on the one or more characteristics input by the user 102, also described with reference to FIG. 4.

In some cases, the system 300 may identify one or more trends (e.g., patterns) associated with the user 102 dreaming. That is, the system 300 may identify one or more trends between multiple dream occurrences (e.g., and/or respective dream characteristics) and at least one of physiological data associated with the user 102, lifestyle data associated with the user 102 (e.g., tags input by the user 102, activities performed by the user 102, current events, food consumed by the user 102, or the like thereof), and respective dream characteristics. For example, the system 300 may identify that the user 102 is likely to dream (e.g., has a higher probability of dreaming) after consuming a high fat dinner based on identifying a threshold quantity of dream occurrences occurring after the user 102 has consumed a high fat dinner. In another example, the system 300 may identify that when a stress level of the user 102 exceeds a threshold stress level, the user is likely to dream about current events or a source of the stress based on identifying the threshold quantity of dream occurrences associated with current event-related characteristics (e.g., as recorded by the user 102) occurring after the user 102 experiences a day where the stress level of the user 102 exceeds the threshold stress level.

FIG. 4 shows an example of a GUI 400 that supports techniques for detecting, recording, and regenerating dreams in accordance with aspects of the present disclosure.

As described previously, with reference to FIG. 3, a system (e.g., the system 300) may detect when a user 102 is dreaming or is likely dreaming and may prompt the user 102, after the user 102 has woken up (e.g., naturally or via alarm), to record information associated with their dream. In such cases, the system may prompt the user 102, via an application page 405-a on the GUI 400 (e.g., on a user device 106), to record the information associated with the dream. For example, the GUI 400 may display a prompt stating “It looks like you may have been dreaming. Would you like to record your dream?” and may enable the user 102 to confirm or deny whether they experienced a dream. In some examples, the system may automatically prompt the user 102 to record a dream. For example, the system may automatically prompt the user 102 to record the dream based on detecting that the user 102 has woken up (e.g., regardless of detection of whether the user 102 dreamt), based on detecting that the user 102 has woken up after dreaming (e.g., based on detecting that the user 102 has dreamt), based on the user 102 picking up the user device 106 within a time range associated with sleep (e.g., between the hours of 10:00 pm to 10:00 am), based on the user 102 opening an application associated with the GUI 400 for a first time during a day (e.g., the user 102 opening the application for the first time in the morning or day, even if no alarms were generated or even if the user 102 did not wake up naturally during the night), based on generating an alarm, or any combination thereof. In some other cases, the system may prompt the user 102 to record the dream based on receiving a user input. For example, the user 102 may wake up (e.g., naturally or based on an alarm) and may input, into the GUI 400, that the user 102 has a dream to record. In some cases, as described previously with FIG. 3, the application page 405-a may prompt the user 102 to enable or disable an alarm feature (e.g., “Dream Alarm”).

To enable the user 102 to record information associated with a dream, an application page 405-b may prompt the user to input one or more characteristics (e.g., characterizations) associated with the dream. For example, the system may prompt the user to select one or more tags (e.g., from multiple tags) associated with the dream. In such cases, the one or more tags may be visual tags (e.g., pictures), textual tags (e.g., words), or both. Additionally, the multiple tags displayed to the user 102 may be specific to the user 102. For example, the multiple tags may include one or more tags previously selected by the user (e.g., associated with previous dreams, tags selected by the user 102 a threshold quantity of times), one or more tags associated with a lifestyle of the user 102 (e.g., activities of the user 102, movies watched by the user 102, stress inducing events associated with the user 102, daytime tags input by the user 102), one or more tags associated with current events near the user 102, or any combination thereof. In such cases, to determine the multiple tags to display to the user 102, the system may receive information from one or more additional applications, such as a calendar application, a movie or TV application (e.g., Hulu, Netflix), a screentime application, or the like thereof, such that the multiple tags may be based on the information received from the one or more additional applications. For example, the system may determine, based on the movie application, that the user 102 watched a scary movie about monsters before bed, such that the system may display a tag stating “Monsters” to the user 102.

Additionally, or alternatively, the application page 405-b may prompt the user 102 to write an entry (e.g., one or more characteristics) about their dream, speak an entry about their dream, or both. For example, the application page 405-b may display a prompt stating “Hit record to tell us about your dream” and may prompt the user 102 to activate a microphone of the user device 106 to enable the user 102 to speak the entry. In some cases, the system may apply a first AI model to the written or spoken entry. That is, the user 102 may speak or write the entry in incomplete sentences (e.g., words, phrase) and the first AI model may modify the entry to include complete sentence with transitionary phrases. For example, the user 102 may wake up in the middle of the night to record a dream and may speak “closet . . . monster . . . run” and the first AI model may output (e.g., based on the input “closet, monster, run”) a sentence stating, “There was a monster in the closet so I ran away.” In such cases, the user 102 may modify or edit an entry output by the first AI model at a time of entry or at a later time (e.g., the next morning). Additionally, or alternatively, the system may auto-record the user 102 in their sleep and use the auto-recording to determine a subset of the multiple tags to display to the user 102, to generate an entry, or both. That is, as described previously with reference to FIG. 2, the system may determine when the user 102 is dreaming and may record audio while the user 102 is dreaming or may record audio when a sound is detected, and may use the audio to determine the subset of the multiple tags to display to the user 102, to generate the entry, or both. For example, the user 102 may talk in their sleep and may state “Catch the dog!” such that the application page 405-b may display tags stating “Dog” and “Run Away” to the user 102.

Additionally, the system may generate, using a second AI model (e.g., the same as or different than the first AI model), one or more of a visual representation, a textual representation, or an auditory representation of the dream based on the information input by the user 102 (e.g., the one or more characteristics, tags, entries) and may display, via an application page 405-c, the visual representation, the textual representation, the auditory representation, or any combination, to the user 102. For example, the one or more characteristics input by the user 102 may include a description of the user 102 driving a car on a sunny day, such that the system may generate, using the second AI model, a short video of the user 102 driving the car on the sunny day. Additionally, the system may prompt the user 102 to input feedback associated with the visual representation, the textual representation, the auditory representation, or any combination thereof. For example, the video generated by the system may depict a red car, however, the user 102 may have dreamt of a blue car, such that the user 102 may indicate the difference in car color to the system. Thus, the system may receive the feedback and may update the visual representation, the textual representation, the auditory representation, or any combination thereof, based on the feedback. For example, the system may update the video to depict the blue car rather than the red car. In some examples, the system may store a log of visual representations, textual representations, and auditory representations displayed to the user 102, such that the user 102 may review previous visual representations, textual representations, and auditory representations at later times.

In some cases, the system may use one or more characteristics of the user 102 (e.g., age, sex, ethnicity, height, weight, hair color, or the like thereof) to generate a depiction of the user 102. In some examples, the one or more characteristics of the user 102 may be based on one or more pictures uploaded by the user 102, one or more user-characteristic tags selected by the user 102, physiological data associated with the user 102, or the like thereof. Similarly, the system may use one or more characteristics associated with other individuals in a life of the user 102 to generate a \depiction of the other individuals. For example, the user 102 may upload a picture of themselves and their partner (e.g., into the system), such that the system may use the picture to generate a representation of the user 102 and a representation of their partner (e.g., when applicable).

A system is described. The system may include a wearable device comprising one or more sensors configured to measure physiological data from a user, a user device communicatively couple with the wearable device, and one or more processors communicatively couple with the user device, the wearable device, or both, wherein the one or more processors are configured to: receive, from the wearable device, first physiological data measured from the user via the wearable device during a first time interval, compute one or more sleep stages of the user during the first time interval based at least in part on the first physiological data, wherein the one or more sleep stages of the user comprises at least a first sleep cycle, detect, based at least in part on the first physiological data, that the user has woken up within a first threshold duration from an end of the first sleep cycle, generate a signal to cause a GUI of the user device associated with the wearable device to display information prompting the user to input one or more characteristics associated with a dream based at least in part on detecting the user has woken up within the first threshold duration from the end of the first sleep cycle, and generate, using a generative artificial intelligence model, one or more of a visual representation, a textual representation, or an auditory representation of the dream based at least in part on the one or more characteristics input by the user.

In some examples of the system described herein, the one or more processors may be further configured to generate an alarm to wake the user within the first threshold duration from the end of the first sleep cycle, wherein the alarm may be an auditory alarm, a haptic alarm, a visual alarm, or any combination thereof, and wherein detecting that the user may have woken up may be based at least in part on generation of the alarm.

In some cases of the system described herein, the first sleep cycle may be a REM cycle. In such cases, to generate the signal to cause the wearable device, the user device, the external device, or any combination thereof, to generate the alarm, the one or more processors may be further configured to generate the signal to cause the wearable device, the user device, the external device, or any combination thereof, to generate the alarm based at least in part on identifying that the user may be within the first threshold duration of the end of the REM cycle, based at least in part on a first duration of the REM cycle being within a second threshold duration of an average REM cycle duration of the user, based at least in part on the first duration of the REM cycle exceeding a third threshold duration, or any combination thereof.

In some examples of the system described herein, identifying that the user may be within the first threshold duration of the end of the REM cycle may be based at least in part on the first physiological data.

In some examples of the system described herein, the one or more processors may be further configured to receive, from the wearable device, second physiological data measured from the user via the wearable device during a second time interval prior to the first time interval, wherein generating the signal to cause the wearable device, the user device, the external device, or any combination thereof, to generate the alarm may be based at least in part on a difference between at least a portion of the first physiological data and at least a portion of the second physiological data exceeding a threshold.

In some examples of the system described herein, the one or more processors may be further configured to receive, from an external device, brain wave data, wherein generating the signal to cause the wearable device, the user device, the external device, or any combination thereof, to generate the alarm may be based at least in part on a frequency of the brain wave data exceeding a threshold.

In some examples of the system described herein, the one or more processors may be further configured to automatically cause the GUI of the user device associated with the wearable device to display the information prompting the user to input one or more characteristics based at least in part on generating the signal to cause the wearable device, the user device, an external device, or any combination thereof, to generate the alarm.

In some examples of the system described herein, generating the signal to cause the wearable device, the user device, an external device, or any combination thereof, to generate the alarm may be based at least in part on satisfaction of one or more criteria, and the one or more criteria may be based at least in part on sleep data associated with the user during a previous night, activity data associated with the user during a previous day, the user being within a second threshold duration of an average wake up time of the user, the user activating a dream characterization feature, the user enabling an alarm feature, or any combination thereof.

In some examples of the system described herein, the one or more processors may be further configured to receive, via the user device, the wearable device, or both, a user input indicating the one or more characteristics associated with the dream, wherein the user input may be a textual input, a verbal input, or both.

In some examples of the system described herein, the textual input may be associated with one or more tags selected from a plurality of tags and the plurality of tags comprise textual tags, visual tags, auditory tags, or any combination thereof.

In some examples of the system described herein, the plurality of tags may be based at least in part on one or more previous characterizations input by the user, the physiological data from the user, a lifestyle of the user, one or more characteristics associated with the user, one or more current events, one or more activities performed by the user during a previous day, or any combination thereof.

In some examples of the system described herein, the one or more processors may be further configured to cause the GUI of the user device associated with the wearable device to display second information prompting the user to input feedback associated with the one or more of the visual representation, the textual representation, or the auditory representation.

In some examples of the system described herein, the one or more processors may be further configured to generate, using the generative artificial intelligence model, one or more of a second visual representation, a second textual representation, or a second auditory representation of the dream based on the feedback input by the user associated with the one or more of the visual representation, the textual representation, or the auditory representation.

In some examples of the system described herein, generating the one or more of the visual representation, the textual representation, or the auditory representation of the dream may be based at least in part on one or more characteristics associated with the user.

In some examples of the system described herein, the one or more processors may be further configured to identify a plurality of dream occurrences based at least in part on a plurality of characterizations input by the user, wherein the plurality of characterizations may be associated with the plurality of dream occurrences, identify one or more trends associated with the user dreaming based at least in part on one or more relationships between the plurality of dream occurrences and a plurality of physiological data, and cause a graphical user interface (GUI) of the user device associated with the wearable device to display information associated with the one or more trends.

In some examples of the system described herein, the one or more processors may be further configured to receive, via the wearable device, the user device, or both, a user input indicating that the user may have dreamt, wherein causing the GUI of the user device associated with the wearable device to display the information prompting the user to input one or more characteristics may be based at least in part on receiving the user input.

In some examples of the system described herein, detecting the user may have woken up may be based at least in part on the first physiological data indicating that the user may be awake.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for recording dream characterizations, comprising:
a wearable device comprising one or more sensors configured to measure physiological data from a user;
a user device communicatively coupled with the wearable device; and
one or more processors communicatively coupled with the user device, the wearable device, or both, wherein the one or more processors are configured to:
receive, from the wearable device, first physiological data measured from the user via the wearable device during a first time interval;
compute one or more sleep stages of the user during the first time interval based at least in part on the first physiological data, wherein the one or more sleep stages of the user comprises at least a first sleep cycle;
detect, based at least in part on the first physiological data, that the user has woken up within a first threshold duration from an end of the first sleep cycle;
generate a signal to cause a graphical user interface (GUI) of the user device associated with the wearable device to display information prompting the user to input one or more characteristics associated with a dream based at least in part on detecting the user has woken up within the first threshold duration from the end of the first sleep cycle; and
generate, using a generative artificial intelligence model, one or more of a visual representation, a textual representation, or an auditory representation of the dream based at least in part on the one or more characteristics input by the user.

2. The system of claim 1, wherein the one or more processors are further configured to:
generate a signal to cause the wearable device, the user device, an external device, or any combination thereof, to generate an alarm to wake the user within the first threshold duration from the end of the first sleep cycle, wherein the alarm is an auditory alarm, a haptic alarm, a visual alarm, or any combination thereof, and wherein detecting that the user has woken up is based at least in part on generation of the alarm.

3. The system of claim 2, wherein the first sleep cycle is a random eye movement (REM) cycle, and wherein, to generate the signal to cause the wearable device, the user device, the external device, or any combination thereof, to generate the alarm, the one or more processors are further configured to:
generate the signal to cause the wearable device, the user device, the external device, or any combination thereof, to generate the alarm based at least in part on identifying that the user is within the first threshold duration of the end of the REM cycle, based at least in part on a first duration of the REM cycle being within a second threshold duration of an average REM cycle duration of the user, based at least in part on the first duration of the REM cycle exceeding a third threshold duration, or any combination thereof.

4. The system of claim 3, wherein identifying that the user is within the first threshold duration of the end of the REM cycle is based at least in part on the first physiological data.

5. The system of claim 2, wherein the one or more processors are further configured to:
receive, from the wearable device, second physiological data measured from the user via the wearable device during a second time interval prior to the first time interval, wherein generating the signal to cause the wearable device, the user device, the external device, or any combination thereof, to generate the alarm is based at least in part on a difference between at least a portion of the first physiological data and at least a portion of the second physiological data exceeding a threshold.

6. The system of claim 2, wherein the one or more processors are further configured to:
receive, from an external device, brain wave data, wherein generating the signal to cause the wearable device, the user device, the external device, or any combination thereof, to generate the alarm is based at least in part on a frequency of the brain wave data exceeding a threshold.

7. The system of claim 2, wherein the one or more processors are configured to:
automatically cause the GUI of the user device associated with the wearable device to display the information prompting the user to input one or more characteristics based at least in part on generating the signal to cause the wearable device, the user device, an external device, or any combination thereof, to generate the alarm.

8. The system of claim 2, wherein generating the signal to cause the wearable device, the user device, an external device, or any combination thereof, to generate the alarm is based at least in part on satisfaction of one or more criteria, and wherein the one or more criteria are based at least in part on sleep data associated with the user during a previous night, activity data associated with the user during a previous day, the user being within a second threshold duration of an average wake up time of the user, the user activating a dream characterization feature, the user enabling an alarm feature, or any combination thereof.

9. The system of claim 1, wherein the one or more processors are further configured to:
receive, via the user device, the wearable device, or both, a user input indicating the one or more characteristics associated with the dream, wherein the user input is a textual input, a verbal input, or both.

10. The system of claim 9, wherein the textual input is associated with one or more tags selected from a plurality of tags, and wherein the plurality of tags comprise textual tags, visual tags, auditory tags, or any combination thereof.

11. The system of claim 10, wherein the plurality of tags are based at least in part on one or more previous characterizations input by the user, the physiological data from the user, a lifestyle of the user, one or more characteristics associated with the user, one or more current events, one or more activities performed by the user during a previous day, or any combination thereof.

12. The system of claim 1, wherein the one or more processors are further configured to:
cause the GUI of the user device associated with the wearable device to display second information prompting the user to input feedback associated with the one or more of the visual representation, the textual representation, or the auditory representation.

13. The system of claim 12, wherein the one or more processors are further configured to:
generate, using the generative artificial intelligence model, one or more of a second visual representation, a second textual representation, or a second auditory representation of the dream based on the feedback input by the user associated with the one or more of the visual representation, the textual representation, or the auditory representation.

14. The system of claim 1, wherein generating the one or more of the visual representation, the textual representation, or the auditory representation of the dream is based at least in part on one or more characteristics associated with the user.

15. The system of claim 1, wherein the one or more processors are further configured to:
identify a plurality of dream occurrences based at least in part on a plurality of characterizations input by the user, wherein the plurality of characterizations are associated with the plurality of dream occurrences;
identify one or more trends associated with the user dreaming based at least in part on one or more relationships between the plurality of dream occurrences and a plurality of physiological data; and
cause a graphical user interface (GUI) of the user device associated with the wearable device to display information associated with the one or more trends.

16. The system of claim 1, wherein the one or more processors are further configured to:
receive, via the wearable device, the user device, or both, a user input indicating that the user has dreamt, wherein causing the GUI of the user device associated with the wearable device to display the information prompting the user to input one or more characteristics is based at least in part on receiving the user input.

17. The system of claim 1, wherein detecting the user has woken up is based at least in part on the first physiological data indicating that the user is awake.
